(19) United States
(12) Patent Application Publication
(10) Pub. No.: US 2024/0374683 A1
(43) Pub. Date: Nov. 14, 2024

(54) FUSION PROTEIN BETWEEN SHORT FORM ROD-DERIVED CONE VIABILITY FACTOR AND A HYDROPHILIC PEPTIDE

(71) Applicant: Pharma Cinq, LLC, Rockville, MD (US)

(72) Inventors: Tianci LUO, Fulton, MD (US); Jun ZHANG, Ellicott City, MD (US)

(21) Appl. No.: 18/785,213

(22) Filed: Jul. 26, 2024

Related U.S. Application Data
(62) Division of application No. 17/172,202, filed on Feb. 10, 2021, now Pat. No. 12,076,367.
Application No. 17/172,202 is a division of application No. 16/301,764, filed on Nov. 15, 2018, now Pat. No. 10,946,063.
(60) Provisional application No. 62/406,552, filed on Oct. 11, 2016.

(51) Int. Cl. A61K 38/17; A61K 47/42; A61K 47/64; A61P 25/14; C07K 14/47; C07K 14/76; C12N 9/02; C12N 15/11; C12N 15/113; C12N 15/85; C12N 15/86

(52) U.S. Cl. CPC A61K 38/1709; A61K 47/42; A61K 47/643; A61P 25/14; C07K 14/47; C07K 14/76; C12N 9/0036; C12N 15/111; C12N 15/113; C12N 15/85; C12N 15/86; C12Y 108/01008; C07K 2319/02; C07K 2319/31; C12N 2750/14143; C12N 2800/22

(57) ABSTRACT

A fusion protein is described, comprising a first N-terminal signal peptide sequence, a second peptide sequence C-terminal to the signal peptide sequence, and a third peptide sequence C-terminal to the second peptide sequence; wherein one of the second peptide sequence and the third peptide sequence is an RdCVF-short peptide sequence and the other is a hydrophilic peptide sequence. After translation the signal peptide is cleaved, leaving a fusion protein comprising the second peptide sequence and the third peptide sequence minus the signal peptide. Also described are nucleic acids and expression vectors encoding the fusion protein, cells comprising the nucleic acid or expression vector, as well as methods of treatment and uses of the fusion protein, nucleic acid, and expression vector. The fusion protein can be produced in vitro by culturing the cells of this invention under conditions allowing for expression and secretion of the encoded fusion protein, and isolating the fusion protein from the cell culture.

### REFERENCE TO SEQUENCE LISTING

The Sequence Listing filed electronically in the form of an Annex C/ST.25 text file and bearing file reference WOC-019PCT is a part of the disclosure.

### BACKGROUND OF THE INVENTION

RdCVF is a thioredoxin-like protein specifically expressed by rod photoreceptor cells in the retina (Léveillard et al. (2004) Nature Genetics 36:755-759 and the supplemental information). Two different RdCVF genes are found in humans and they are designated RdCVF1 and RdCVF2. Both RdCVF genes encode two products via alternative splicing: a full length protein and a C-terminal post-transcriptionally truncated protein, known as RdCVF-long (RdCVFL) and RdCVF-short (RdCVFS), respectively. The nucleoredoxin-like-1 gene (Nxn11) encodes a long and a short form RdCVF by alternative splicing mechanism. Nxn11 knockout results in progressive loss of rods and cones in mice, suggesting at the genetic level this gene is fundamentally essential for survival of photoreceptor cells and maintaining proper retina physiology and function.

RdCVFS is described as a secreted trophic factor for promoting cone survival, and RdCVFL as a redox-active enzyme that interacts with intracellular proteins (Léveillard et al. (2010) Sci Transl Med. 2(26): 26ps 16). For example, tau is described as a binding partner for RdCVF-L and tau is exclusively intracellular (Fridlich et al. (2009) Molecular & Cellular Proteomics 8(6):1206-18).

Individuals suffering from some retinal dystrophies were found to have lower levels of RdCVF protein in their eyes than did individuals without retinal dystrophies (PCT Publication WO 02/081513).

It has been demonstrated that different forms of RdCVF protein can promote cone photoreceptor cell survival in vitro and in vivo. For example, intraocular injections of the short form of human RdCVF1 (RdCVF1S) protein not only rescued cone cells from degeneration but also preserved their function in animal models of inherited retinal degeneration (Yang et al. (2009) Mol Therapy 17:787-795). However, demonstration of the in vivo cone cell protective effect of this protein required multiple intraocular injections.

Retinitis pigmentosa (RP) is retinal degenerative eye disease characterized by progressive rod degeneration followed by secondary loss of cones. RP is the leading cause of inherited blindness, affecting approximately 100,000 patients with 2,000 new cases per year in US alone. RP affects all ethnicities. More than 1.5 million people are affected by RP worldwide. Unfortunately for the patients, there is no effective therapy or approved therapy for RP. Therefore, RP remains an urgent unmet medical need.

Since RP is a chronic retinal degenerative disease with a clinical course over years to decades, gene therapy could be ideal for RP indication by expressing RdCVF in the retina constitutively. For acute emergency indications such as retinal detachment, protein therapy could be beneficial. That is to use recombinant RdCVF protein to protect photoreceptors from dying before the retina could be re-connected to the back of the eye, i.e., retinal pigment epithelium and choroid layers. Unfortunately, scientists in the field have encountered considerable difficulties to effectively express and secrete RdCVF protein, especially the short form RdCVF. See, for example, U.S. Patent Publication No. 20110034546, paragraph [0004].

### SUMMARY OF THE INVENTION

This invention provides a fusion protein comprising a first N-terminal signal peptide sequence, a second peptide sequence C-terminal to the signal peptide sequence, and a third peptide sequence C-terminal to the second peptide sequence; wherein one of the second peptide sequence and the third peptide sequence is an RdCVF-short peptide sequence and the other is a hydrophilic peptide sequence. After translation the signal peptide is cleaved in the endoplasmic reticulum, leaving a fusion protein comprising the second peptide sequence and the third peptide sequence minus the signal peptide.

Therefore this invention also provides a fusion protein comprising a second peptide sequence and a third peptide sequence C-terminal to the second peptide sequence; wherein one of the second peptide sequence and the third peptide sequence is an RdCVF-short peptide sequence and the other is a hydrophilic peptide sequence. This invention also provides nucleic acids and expression vectors encoding the fusion protein, cells comprising the nucleic acid or expression vector, as well as methods of treatment and uses of the fusion protein, nucleic acid, and expression vector. This invention also provides a method for producing the fusion protein comprising culturing the cells of this invention under conditions allowing for expression and secretion of the encoded fusion protein, and isolating the fusion protein from the cell culture.

### BRIEF DESCRIPTION OF THE FIGURES

FIG. 1A shows amino acid sequence of human short form rod-derived cone viability factor. The amino acid composition for the human short RdCVF is highly hydrophobic. 38.5% of amino acids are hydrophobic in short RdCVF. Underlined amino acids are hydrophobic.

FIG. 1B shows amino acid sequence of human long form rod-derived cone viability factor. 25% of amino acids are hydrophobic at the C-terminus of long RdCVF (Underlined amino acids are hydrophobic at the C-terminus of the long form RdCVF)

FIG. 2. Fusion protein between short RdCVF and hydrophilic domain. FIG. 2 shows schematic representations of two human short RdCVF fusion proteins. Human short RdCVF is fused with a hydrophilic domain at its N-terminus or C-terminus. The fusion protein has a signal peptide to facilitate secretion from cells.

FIG. 3. Expression and secretion of novel fusion proteins between human albumin and human short RdCVF.

FIG. 3 shows a Western blot analysis of human short RdCVF and human albumin fusion proteins.

Lane 1:30 μL of cell culture medium from human 293 cells transduced with AAV-GFP, an AAV vector encoding GFP as a control;

Lane 2:30 μL of cell culture medium from human 293 cells transduced with AAV-ALB-RdCVFS, an AAV vector encoding the short form human RdCVF fused with human albumin at the N-terminus of short RdCVF;

Lane 3:30 μL of cell culture medium from human 293 cells transduced with AAV-RdCVFS-ALB, an AAV vector encoding the short form human RdCVF fused with human albumin at the C-terminus of short RdCVF.

The numbers on the left indicate molecular weight marker in KDa.

### BRIEF DESCRIPTION OF THE SEQUENCE LISTING

SEQ ID NO: 1. Amino acid sequence of a fusion protein between human albumin and short RdCVF with human albumin at the N-terminus of the fusion protein (ALB-RdCVFS)

SEQ ID NO: 2. Nucleotide Sequence Encoding the Human Short RdCVF Fused with Human Albumin at N-terminus (ALB-RdCVFS)

SEQ ID NO: 3. Amino acid sequence of a fusion protein between human albumin and short RdCVF with human albumin at the C-terminus of the fusion protein (RdCVFS-ALB). There is a four amino acid spacer between RdCVFS and the human albumin. The first 21 amino acids from the N-terminus are signal sequence from mouse Igk. There is a 14 amino acid spacer (linker) between the signal sequence and RdCVFS.

SEQ ID NO:4. Nucleotide Sequence Encoding the Human Short RdCVF Fused with Human Albumin at C-terminus (RdCVFS-ALB)

SEQ ID NO: 5. Amino acid sequence of human short form rod-derived cone viability factor.

SEQ ID NO:6. Amino acid sequence of human long form rod-derived cone viability factor.

### DETAILED DESCRIPTION OF THE INVENTION

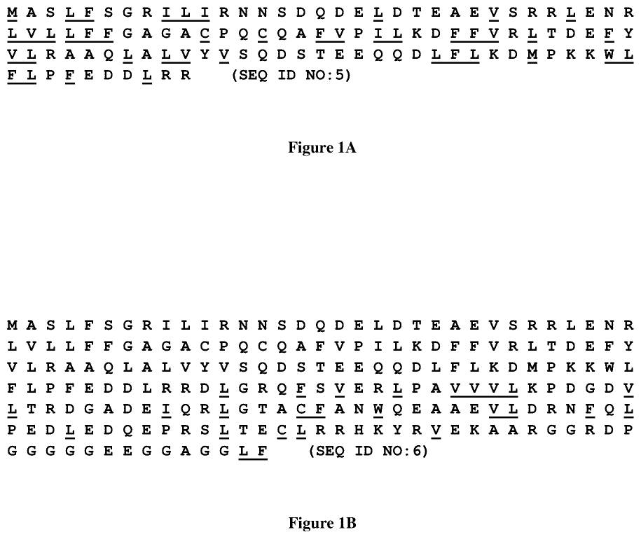

Without wishing to be bound by theory, the difficulty to express and secrete the short form RdCVF could potentially be due to its high hydrophobic amino acid composition. Careful analysis of the amino acid composition of both the short form and the long form of human RdCVF proteins revealed that the short form RdCVF protein is extremely hydrophobic (FIGS. 1A and 1B). Forty two amino acids out of 109 (38.5%) are hydrophobic amino acids in short RdCVF. There is one stretch of six hydrophobic amino acids, one stretch of four hydrophobic amino acid, and two stretches of three hydrophobic amino acids each. It is highly likely that the high percentage of hydrophobic amino acid composition makes the short RdCVF very difficult to be expressed and secreted efficiently in vitro and in vivo from mammalian cells as it is more likely to stick to lipid membranes via hydrophobic-hydrophobic interactions. Interestingly, although the N-terminal 109 amino acids in the long form RdCVF are identical to the entire short RdCVF, the C-terminal 103 amino acids of the long RdCVF is not hydrophobic with only 25% of amino acids being hydrophobic (26 out of 103). The longest hydrophobic amino acids stretch in this C-terminus of the long RdCVF is only four amino acids long. There is no stretch of three hydrophobic amino acids. The relatively hydrophilic nature in the C-terminus of long RdCVF may play an important role in reducing the overall hydrophobicity of the long RdCVF.

In one embodiment of the fusion protein of this invention, the second peptide sequence is an RdCVF-short peptide sequence and the third peptide sequence is a hydrophilic peptide sequence. In another embodiment the second peptide sequence is a hydrophilic peptide sequence and the third peptide sequence is an RdCVF-short peptide sequence.

There should be an N-terminal signal peptide sequence that will facilitate the secretion of the fusion protein out of the cells that express this fusion protein. Any signal peptide that enables the secretion of a protein from mammalian cells can be utilized. In further embodiments, the signal peptide sequence is selected from the group consisting of an Igk signal peptide sequence and an albumin signal peptide sequence. In more specific embodiments the IgK signal peptide sequence is a mouse IgK signal peptide sequence and the albumin signal peptide sequence is a human albumin signal peptide sequence.

In a preferred embodiment of this invention the RdCVF-short peptide sequence is a human RdCVF-short peptide sequence. Examples of suitable RdCVF-short peptide sequences include an RdCVF1-short peptide sequence, an RdCVF2-short peptide sequence, and an RdCVF-short peptide sequence that differs from a corresponding wild-type sequence, for example by one or more conservative amino acid substitutions.

In accordance with this invention any hydrophilic peptide sequence can be utilized provided that lowers the hydrophobicity index of the fusion protein compared to the RdCVF-short peptide sequence without the hydrophilic peptide sequence. In one embodiment of this invention the fusion protein, including any signal peptide sequence if present, has a hydrophobicity index less than negative 0.20, more preferably less than negative 0.30.

Preferably the hydrophilic peptide sequence is not immunogenic in humans, does not present any other negative effect on human retinal physiology or normal retinal function, and does not affect short RdCVF's biological function. The hydrophilic peptide sequence can be a hydrophilic protein, a hydrophilic protein domain, a hydrophilic oligopeptide, or a hydrophilic polypeptide. More than one hydrophilic domain can be the candidate as the hydrophilic fusion partner with short RdCVF. In one embodiment of this invention the hydrophilic peptide sequence is an albumin, for example a human albumin.

In accordance with the fusion protein of this invention there can optionally be a spacer of one or more amino acids between the first peptide sequence and the second peptide sequence, between the second peptide sequence and the third peptide sequence, or both. In one embodiment of this invention there is no spacer between the first peptide sequence and the second peptide sequence, or in other words the first peptide sequence is covalently bonded to the second peptide sequence by a single peptide bond. In another embodiment there is a spacer between the first peptide sequence and the second peptide sequence. In another embodiment of this invention there is no spacer between the second peptide sequence and the third peptide sequence, or in other words the second peptide sequence is covalently bonded to the third peptide sequence by a single peptide bond. In another embodiment there is a spacer between the second peptide sequence and the third peptide sequence. There is in principle no limitation on the size of the spacers. In an embodiment of this invention the spacer between the first and second peptide sequence has two to fourteen amino acids. In another embodiment the spacer between the second and third peptide sequences has from two to fourteen amino acids, more specifically between two and four amino acids.

One embodiment of the fusion protein coding sequence of this invention further comprises a polyadenylation signal C-terminal to the third peptide sequence coding sequence. The polyadenylation signal (Poly A) can be any Poly A.

In one embodiment of the fusion protein of this invention, the first peptide sequence is a human albumin signal sequence, the second peptide sequence is a human albumin, and the third peptide sequence is an RdCVF-short sequence. In a more specific embodiment the fusion protein has the sequence (SEQ ID NO:1) or amino acids 25-717 of (SEQ ID NO:1). In another embodiment of the fusion protein of this invention the first peptide sequence is an Igk signal sequence, the second peptide sequence is an RdCVF-short sequence, and the third peptide sequence is a human albumin. In a more specific embodiment the fusion protein has the sequence (SEQ ID NO:3) or amino acids 22-732 of (SEQ ID NO:3).

In an embodiment of the fusion protein of this invention, wherein one, two, or all of the signal peptide sequence, the RdCVF-short peptide sequence, and the hydrophilic peptide sequence differs from a corresponding wild-type sequence. In a more specific embodiment the differences comprise one or more conservative amino acid substitutions. And in a still more specific embodiment the amino acid substitutions are limited one or more conservative amino acid substitutions.

The fusion protein in accordance with this invention can be glycosylated or not glycosylated. Generally speaking, glycosylation is beneficial for a protein's stability and solubility. In one embodiment the fusion protein expressed in cells transduced by an expression vector in accordance with this invention is glycosylated and is glycosylated after secretion from the cells.

In an embodiment the nucleic acid of this invention is DNA. In another embodiment the coding sequence for one, two, or all of the signal peptide sequence, the RdCVF-short peptide sequence and the hydrophilic peptide sequence is recoded compared to a corresponding wild-type sequence. In a more specific embodiment the coding sequence for the RdCVF-short peptide sequence is recoded. The nucleic acid of this invention can optionally comprise one or more introns, either between the first and the second peptide sequences, between the second and third peptide sequences, or elsewhere. In one embodiment of this invention the nucleic acid encodes a fusion protein having the sequence (SEQ ID NO:1), for example a nucleic acid having the sequence (SEQ ID NO:2). In another embodiment the nucleic acid encodes a fusion protein having the sequence (SEQ ID NO:3), for example a nucleic acid having the sequence (SEQ ID NO: 4). Other nucleic acid sequences can readily be envisioned in view of the degeneracy of the genetic code.

An embodiment of this invention is an expression vector comprising the nucleic acid described above operatively linked to a control sequence, for example a promoter. The promoter driving the RdCVFS fusion protein can be any promoter and is not limited to CMV promoter. When there is an intron between the promoter and the fusion protein coding sequence, any suitable and conventional intron can be utilized. For example, β-globin intron is suitable.

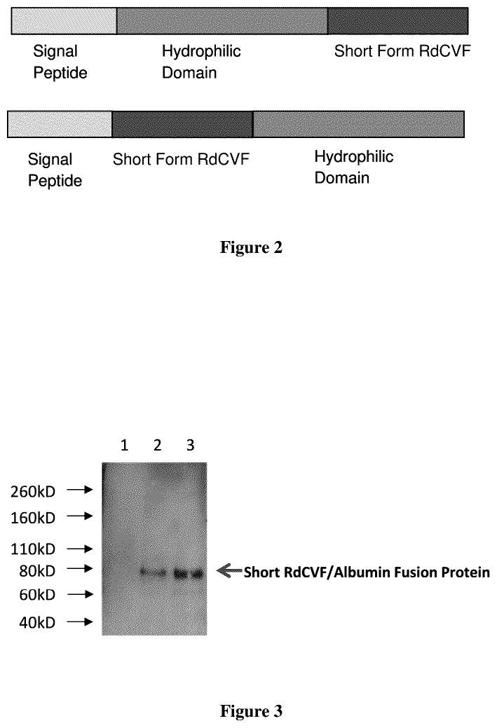

In the experiments two fusion proteins were created by fusing human short RdCVF with a hydrophilic domain (See FIG. 2 for schematic). One was human short RdCVF fused with human albumin at the N-terminus with an albumin signal peptide at the N-terminus of the fusion protein (SEQ ID NO:1). The expression construct to express and secrete this fusion protein was engineered in the context of an AAV vector, designated rAAV-ALB-RdCVFS. This vector encoded a codon-optimized (recoded) human short RdCVF fused with human albumin at the N-terminus (SED ID NO: 2). The fusion protein was named ALB-RdCVFS. A coding sequence for human albumin signal peptide was also incorporated into the fusion protein expression construct upstream of the fusion protein coding sequence. The expression construct further contained a CMV promoter and an intron that linked to the fusion protein coding sequence. The expression construct further contained a polyadenylation signal at the C-terminus of the fusion protein coding sequence. The entire expression cassette was cloned into an AAV expression plasmid and the plasmid was subjected to DNA sequencing to confirm the integrity of the expression construct.

The other exemplified fusion protein was human short RdCVF fused with human albumin at the C-terminus with a mouse Igk signal peptide at the N-terminus of the fusion protein (SEQ ID NO:3). The expression construct to express and secrete this fusion protein was engineered in the context of an AAV vector, designated rAAV-RdCVFS-ALB. This vector encoded a codon-optimized (recoded) human short RdCVF fused with human albumin at the C-terminus (SEQ ID NO:4). The fusion protein was named RdCVFS-ALB. A coding sequence for a modified mouse Igk signal peptide was also incorporated into the fusion protein expression construct upstream of the fusion protein coding sequence. The expression construct further contained a CMV promoter and an intron that linked to the fusion protein coding sequence. The expression construct further contained a polyadenylation signal at the C-terminus of the fusion protein coding sequence. The entire expression cassette was cloned into an AAV expression plasmid and the plasmid was subjected to DNA sequencing to confirm the integrity of the expression construct.

There are a total of 20 naturally-occurring amino acids. Some of them are hydrophobic and some of them are hydrophilic. The hydrophobicity index of an amino acid is a number representing the hydrophobic or hydrophilic properties of its sidechain. The larger the number is, the more hydrophobic the amino acid (Table 1). The most hydrophobic amino acids are isoleucine (4.5) and valine (4.2). The most hydrophilic ones are arginine (−4.5) and lysine (−3.9). Although a protein's hydrophobic or hydrophilic nature depends on what amino acids it is made of, as well as the secondary, tertiary and quaternary structure, the hydrophobicity index of a protein can have predictive value for a protein's hydrophobicity.

TABLE 1
Hydrophobicity Value
of Amino Acids
Ala: 1.800
Arg: −4.500
Asn: −3.500
Asp: −3.500
Cys: 2.500
Gln: −3.500
Glu: −3.500
Gly: −0.400
His: −3.200
Ile: 4.500
Leu: 3.800
Lys: −3.900
Met: 1.900
Phe: 2.800
Pro: −1.600
Ser: −0.800
Thr: −0.700
Trp: −0.900
Tyr: −1.300
Val: 4.200

The publicly-available GPMAW program was used to calculate the hydrophobicity index for the short RdCVF, and the two short RdCVF and albumin fusion proteins. The hydrophobicity index for the native short form of human RdCVF (RdCVFS) is −0.12. After fusing the human albumin to the N-terminus of RdCVFS, the resulting fusion protein, ALB-RdCVFS had its hydrophobicity index reduced from −0.12 to −0.32 (including the signal peptide), a 266.7% reduction in hydrophobicity index. After fusing the human albumin to the C-terminus of RdCVFS, the resulting fusion protein, RdCVFS-ALB had its hydrophobicity index reduced from −0.12 to −0.33 (including the signal peptide), a 275% decrease in hydrophobicity index. The dramatic reduction in hydrophobicity index may have contributed to efficient expression and secretion of the fusion proteins.

Furthermore, the fusion protein coding sequence has been cloned into an AAV-2 expression construct, the data included here clearly showed that this novel fusion protein was efficiently expressed as detected by Western blot with RdCVF specific antibodies (FIG. 3). The fusion partner for the short RdCVF is not limited to human albumin. Any hydrophilic protein or hydrophilic protein domain or hydrophilic peptide could be used to create a fusion protein with short RdCVF to reduce the protein's hydrophobicity. A preferred hydrophilic domain is non-immunogenic to human.

The protein can be encoded and delivered by a gene therapy vector such as AAV (Adeno-associated Virus) vector, Lentiviral vector, synthetic vector, adenoviral vector, retroviral vector, naked DNA, nanoparticles, etc. Alternatively, the fusion protein can be delivered to the retina as a recombinant protein. The hydrophilic fusion domain should not present any negative effect on human retinal physiology or normal retinal function, and should not affect short RdCVF's biological function. Potentially, more than one hydrophilic domain can be the candidate as the hydrophilic fusion partner with short RdCVF. In the examples, human albumin was used as the hydrophilic domain to serve as a fusion partner for human short RdCVF.

This invention provides a pharmaceutical composition comprising: (i) a component selected from the group consisting of a fusion protein, nucleic acid, expression vector, or cell of this invention; and (ii) a pharmaceutically acceptable carrier.

This invention provides a method for treating a condition amenable to such treatment in a mammalian patient, the treatment comprising administering to the patient an effective amount of the protein, the nucleic acid, the vector, the cell, or the pharmaceutical composition described herein, thereby treating the condition in the patient. In embodiments of this invention the condition is selected from the group consisting of retinal dystrophy, Stargardt's disease, retinitis pigmentosa, dry age-related macular degeneration (dry AMD), geographic atrophy (advanced stage of dry AMD), wet age-related macular degeneration (wet AMD), glaucoma with or without ocular hypertension, diabetic retinopathy, Bardet-Biedel syndrome, Bassen-Kornzweig syndrome, Best disease, choroidema, gyrate atrophy, congenital amaurosis, refsun syndrome, Usher syndrome, thyroid related eye disease, Grave's disease, a disease associated with retinal pigmented epithelial cells, anterior segment disease, lens disease/cataracts, an eye cup disorder, uveitis, Alzheimer's disease, Huntington's disease, Parkinson's disease, and an olfactory disease. Any conventional route of administration can be utilized, for example, injection to the eye, intravenous injection, or other systemic administration. In an embodiment of the method of this invention the condition is an ocular condition and the administration is selected from the group consisting of subretinal injection and intravitreal injection. In a more specific embodiment the patient is a human patient.

This invention provides a method of protecting ocular photoreceptor cells in a patient, comprising administering to the eye of the patient an effective amount of the fusion protein, nucleic acid, vector, cells, or pharmaceutical composition of this invention, thereby protecting the ocular photoreceptor cells in the patient. In a more specific embodiment the administration is selected from the group consisting of subretinal injection and intravitreal injection. In a more specific embodiment the patient is a human patient.

The invention is now described with reference to the following examples. These examples are provided for the purpose of illustration only and the invention should in no way be construed as being limited to these examples but rather should be construed to encompass any and all variations which become evident as a result of the teachings provided herein.

Whereas, particular embodiments of the invention have been described herein for purposes of description, it will be appreciated by those skilled in the art that numerous variations of the details may be made without departing from the invention as described in the appended claims.

All publications, patents and patent applications mentioned in this specification are herein incorporated by reference in their entirety into the specification to the same extent as if each individual publication, patent or patent application was specifically and individually indicated to be incorporated herein by reference. Also incorporated by reference is any supplemental information that was published along with any of the aforementioned publications, patents and patent applications. For example, some journal articles are published with supplemental information that is typically available online.

### EXAMPLES

### Example 1 Generation of rAAV Vectors Encoding Human Short form Rod-Derived Cone Viability Factor and albumin Fusion Proteins

### Plasmid Cloning

The cDNA of the codon-optimized human short form of the RdCVF protein fused with human albumin at it N-terminus was synthesized by GENEART® (a fee-for-service company) and cloned into the adeno-associated virus vector plasmid pAAV-MCS (Cell Biolabs, San Diego, CA), creating the plasmid pAAV-ALB-RdCVFS. The signal peptide from human albumin was also incorporated at the upstream of the RdCVF and albumin fusion protein coding sequence.

The pAAV-ALB-RdCVFS plasmid contains the following features between AAV-ITRs:

CMV promoter—β-globin intron—Signal sequence-Albumin-short RdCVF—Poly A

The resulting amino acid sequence for this RdCVF and albumin fusion protein is shown in SEQ ID NO: 1.

The cDNA of the codon-optimized human short form of the RdCVF protein fused with human albumin at it C-terminus was synthesized by GENEART® (a fee-for-service company) and cloned into the adeno-associated virus vector plasmid pAAV-MCS (Cell Biolabs, San Diego, CA), creating the plasmid pAAV-RdCVFS-ALB. This signal peptide from mouse Igk was also incorporated at the upstream of the RdCVF and albumin fusion protein coding sequence.

The pAAV-RdCVFS-ALB plasmid contains the following features between AAV-ITRs:

CMV promoter—β-globin intron—Signal sequence-short RdCVF-Albumin—Poly A

The resulting amino acid sequence for this RdCVF and albumin fusion protein is shown in SEQ ID NO: 3.

### Production and Purification of Recombinant AAV-ALB-RdCVFS and AAV-RdCVFS-ALB Vectors

Plasmids pAAV-ALB-RdCVFS or pAAV-RdCVFS-ALB, pHELPER (Cell BioLabs, Catalog No. 340202), and pRC2 (Cell BioLabs, Catalog No. 340201) were transformed into DH10B competent bacteria cells (Invitrogen, Catalog No. 18297-010) and scaled up using the Qiagen EndoFree Plasmid Maxi Kit or EndoFree Plasmid Mega Kit according to the manufacturer's instructions. The plasmid concentrations were determined using a Beckman DU-600 spectrophotometer. Each plasmid's identity was confirmed by restriction digests and DNA sequencing analysis.

To produce rAAV-ALB-RdCVFS or rAAV-RdCVFS-ALB vector, 293AAV cells (Cell BioLabs, Catalog No. AAV-100) were seeded at 4 million cells per 15 cm dish in CDMEM (DMEM supplemented with 10% FBS, 1% Glutamine, 1% non-essential amino acids, and 1% Penicillin/Streptomycin). The following day the medium was replaced with 25 mL fresh cDMEM. Two hours later the transfection was performed. Water (57.4 mL) was mixed with 1.3 mg pHELPER, 650 μg pRC2, 650 μg pAAV-ALB-RdCVFS or pAAV-RdCVFS-ALB, and 8.1 mL 2 M CaCl₂ (water/plasmid/CaCl₂ mix). A 12.5 mL volume of 2×HBS (Lonza, Sku:RR07005) was transferred into each of five 50 mL conical tubes. While vortexing, 12.5 mL of the water/plasmid/CaCl₂ mix was slowly added to each of the conical tubes containing 2×HBS. After a 5-minute incubation, 2.5 mL of the suspension was added to each cell culture dish containing the 293AAV cells.

The following day the medium was replaced with 25 mL new cDMEM medium per dish. Two days later the cells were harvested using a cell lifter and the cell/medium mix was transferred into 250 ml conical tubes. The samples were centrifuged at 3,000 rpm for 15 minutes at 4° C., the supernatant was discarded and the cell pellets resuspended in 110 mL DMEM. The resuspended cell samples were aliquoted (30 mL) into 50 mL conical tubes and a freeze/thaw/freeze step was performed using ethanol/dry ice bath and 37° C. water bath. The tubes were stored at −80° C. until further process of the material. The same process was employed to produce rAAV-GFP control vector, substituting the plasmid pAAV-ALB-RdCVFS with the plasmid pAAV-GFP (Cell BioLabs Catalog No. AAV-400).

To purify the rAAV-ALB-RdCVFS vector, four 50 ml conical tubes containing the vector from the freeze/thaw/freeze step was thawed at 37° C. in a water bath. Forty microliters of BENZONASE® (nuclease) (Sigma, Catalog No. E8263-25kU) was added to each tube which was then incubated at 37° C. for 30 minutes. The tubes were centrifuged for 10 minutes at 3,000 rpm and the supernatants were transferred into a 500 mL bottle. Six milliliters of a 10% sodium deoxycholate solution (8.2 g in 82 mL water) was added. The sample was briefly mixed and incubated at 37° C. for 30 minutes. The suspension was filtered using Z5 μm filters. Subsequently, another filtration step using 0.8 μm filter was performed. A heparin agarose column (8 mL) (Sigma, Catalog No. H6508-25 mL) was prepared and the column was equilibrated with 48 mL phosphate buffered saline (PBS) (Invitrogen, Catalog No. 10010-049). The filtered cell lysate was loaded onto the column and the column was washed with 40 mL washing buffer (20 mL 5 M NaCl, 980 mL PBS). The vector was eluted using 15 mL elution buffer (80 mL 5 M NaCl, 920 mL PBS) and collected in a new 50 ml conical tube.

The vector was concentrated by centrifugal filtration. An AMICON ULTRA-15 centrifugational filter unit (Millipore, Catalog No. UFC910024) was rinsed once with PBS and the eluted sample was added to the device. Centrifugation was performed in a Beckman Allegro 6KR centrifuge at 2,200 rpm, 22° C., until the sample was concentrated to a 1-2 mL volume. A 15 mL volume of PBS was added and the centrifugation was repeated until the sample volume was ≤1 mL. The purified vector was collected and the filter walls rinsed with 100 L of PBS. The sample was mixed and 30 μL aliquots of the vector were stored at −80° C. in 600 μL conical tubes until use.

This process was repeated to purify the rAAV-RdCVFS-ALB and rAAV-GFP vectors.

### Example 2: Expression and Secretion of Human Short RdCVF and Human albumin Fusion Proteins Mediated by rAAV Vectors

Western blot analysis using a 4-20% SDS-PAGE gel was used to detect RdCVF and albumin fusion protein expression using standard techniques. As a control, a 5 μL volume of MAGICMARK™ XP Western Protein Standard (Invitrogen, Catalog No. LC5602) was added to the outer wells. 30 μL of cell culture medium from each rAAV vector transduced human 293 cells mixed with protein sample buffer was added to each well. The gel was run at 200 V until the dye reached the bottom of the gel. Western blot analysis was performed with a VECTASTAIN® ABC-Amp Western blot analysis kit by Vector Laboratories, according to a modified version of the manufacturer's instructions. The SDS-PAGE was equilibrated in transfer buffer for 20 min and proteins separated by SDS-PAGE were transferred onto a nitrocellulose membrane using a Trans Blot Semi-Dry Transfer Cell at 20 V for 40 minutes. Once the transfer was completed, the membrane was blocked in 200 mL of 1× casein solution with gentle agitation on a rocker platform for at least two hours at room temperature (RT) or at 4° C. over night. The membrane was incubated with 50 mL 1× cascin solution containing rabbit anti-RdCVF protein specific antibody (primary antibody, generated by Covance (Denver, PA) diluted 1:2,000 with gentle agitation at 4° C. overnight or 1 hour at room temperature. The membrane was washed with 30 mL of a 1× casein solution 4 times for 5 minutes each at RT with gentle agitation. The membrane was incubated with 30 mL of biotinylated goat anti-rabbit IgG (secondary antibody) diluted 1:10,000 in 1× casein solution for 1 hour at RT with gentle agitation. The membrane was washed in 30 mL of 1× casein solution 3 times for 5 minutes each at RT with gentle agitation. The membrane was incubated for 45 minutes in Vectastain ABC-AmP in 50 mL of 1× casein containing 100 μL of Reagent A and 100 μL of Reagent B. The membrane was washed in 30 mL of 1× casein solution 3 times for 5 minutes cach at RT with gentle agitation.

The membrane was incubated in Tris, pH 9.5. The chemiluminescent signal was acquired using 6 mL of DUOLOX Substrate (Vector Laboratories, Catalog No. SK 6605) and exposing the membrane to KODAK BIOMAX MS X-ray film (Kodak Carestream Health, Catalog No. 8572786) in a film cassette for 10 seconds to 5 minutes followed by development of the film using KODAK Developer solution (Kodak GBX, Catalog No. 1900984) and KODAK Fixer solution.

As shown in FIG. 3, cell culture medium from both rAAV-ALB-RdCVFS and rAAV-RdCVFS-ALB transduced human 293 cells contained a band at molecular weight approximately 80 KDa that specifically reacted with rabbit anti-RdCVF antibodies. This band was not detected in the cell culture medium from rAAV-GFP control vector transduced cells. The data suggest both rAAV-ALB-RdCVFS and rAAV-RdCVFS-ALB vectors mediated human short RdCVF and albumin fusion protein expression and secretion in human cells.

The rAAV-ALB-RdCVFS or rAAV-RdCVFS-ALB vectors can be used for intraocular administration to treat the diseases listed above. Specifically, the vectors can be delivered via subretinal injection or intravitreal injection.

### SUMMARY

The recombinant AAV vector encoding a codon-optimized (recoded) human short RdCVF fused with human albumin fusion protein coding sequence at its N-terminus was able to mediate the fusion protein expression in and secretion from human cells. The recombinant AAV vector encoding a codon-optimized human short RdCVF fused with human albumin fusion protein at its C-terminus was able to mediate the fusion protein expression in and secretion from human cells.

What is claimed is:

1. A fusion protein comprising: (a) a first N-terminal signal peptide sequence, a second peptide sequence C-terminal to the signal peptide sequence, and a third peptide sequence C-terminal to the second peptide sequence; or (b) a second peptide sequence and a third peptide sequence C-terminal to the second peptide sequence;
wherein one of the second peptide sequence and the third peptide sequence is an RdCVF-short peptide sequence and the other is a hydrophilic peptide sequence.

2. The fusion protein of claim 1, wherein the second peptide sequence is an RdCVF-short peptide sequence and the third peptide sequence is a hydrophilic peptide sequence.

3. The fusion protein of claim 1, wherein the second peptide sequence is a hydrophilic peptide sequence and the third peptide sequence is an RdCVF-short peptide sequence.

4. The fusion protein of claim 1, wherein the signal peptide sequence is a human signal peptide sequence.

5. The fusion protein of claim 1, wherein the signal peptide sequence is selected from the group consisting of an Igk signal peptide sequence and an albumin signal peptide sequence.

6. The fusion protein of claim 5, wherein the albumin signal peptide sequence is a human albumin signal peptide sequence.

7. The fusion protein of claim 1, wherein the RdCVF-short peptide sequence is a human RdCVF-short peptide sequence.

8. The fusion protein of claim 7, wherein the RdCVF-short peptide sequence is selected from the group consisting of an RdCVF1-short peptide sequence and an RdCVF2-short peptide sequence.

9. The fusion protein of claim 1, wherein the hydrophilic peptide sequence is selected from the group consisting of a hydrophilic protein, a hydrophilic protein domain, a hydrophilic oligopeptide, and a hydrophilic polypeptide.

10. The fusion protein of claim 9, wherein the hydrophilic peptide sequence is an albumin.

11. The fusion protein of claim 10, wherein the albumin is a human albumin.

12. The fusion protein of claim 1, wherein the hydrophilic peptide sequence is not immunogenic to humans.

13. The fusion protein of claim 1, wherein the fusion protein has a hydrophobicity index less than negative 0.20.

14. The fusion protein of claim 13, wherein the fusion protein has a hydrophobicity index less than negative 0.30.

15. The fusion protein of claim 1, wherein the first peptide sequence is covalently bonded to the second peptide sequence by a single peptide bond.

16. The fusion protein of claim 1, wherein there is a spacer between the first peptide sequence and the second peptide sequence.

17. The fusion protein of claim 16, wherein the spacer between the first and second peptide sequences has from two to fourteen amino acids.

18. The fusion protein of claim 1, wherein the second peptide sequence is covalently bonded to the third peptide sequence by a single peptide bond.

19. The fusion protein of claim 1, wherein there is a spacer between the second peptide sequence and the third peptide sequence.

20. The fusion protein of claim 19, wherein the spacer between the second and third peptide sequences has from two to four amino acids.

21. The fusion protein of claim 1, further comprising a polyadenylation signal C-terminal to the third peptide sequence.

22. The fusion protein of claim 1, wherein the first peptide sequence is a human albumin signal sequence, the second peptide sequence is a human albumin, and the third peptide sequence is an RdCVF-short sequence.

23. The fusion protein of claim 22, wherein the fusion protein has the sequence (SEQ ID NO: 1) or amino acids 25-717 of (SEQ ID NO:1).

24. The fusion protein of claim 1, wherein the first peptide sequence is an Igk signal sequence, the second peptide sequence is an RdCVF-short sequence, and the third peptide sequence is a human albumin.

25. The fusion protein of claim 24, wherein the fusion protein has the sequence (SEQ ID NO: 3) or amino acids 22-732 of (SEQ ID NO:3).

26. The fusion protein of claim 1, wherein one, two, or all of the signal peptide sequence, the RdCVF-short peptide sequence, and the hydrophilic peptide sequence differs from a corresponding wild-type sequence.

27. The fusion protein of claim 26, wherein one, two, or all of the signal peptide sequence, the RdCVF-short peptide sequence, and the hydrophilic peptide sequence differs from the corresponding wild-type sequence by one or more conservative amino acid substitutions.

28. A nucleic acid encoding the fusion protein of any one of claims 1 through 27.

29. The nucleic acid of claim 28, wherein the nucleic acid is DNA.

30. The nucleic acid of claim 28, wherein the coding sequence for one, two, or all of the signal peptide sequence, the RdCVF-short peptide sequence and the hydrophilic peptide sequence is recoded compared to a corresponding wild-type sequence.

31. The nucleic acid of claim 30, wherein the coding sequence for the RdCVF-short peptide sequence is recoded.

32. The nucleic acid of claim 29, further comprising one or more introns.

33. The nucleic acid of claim 28, encoding a fusion protein having the sequence (SEQ ID NO: 1).

34. The nucleic acid of claim 33, having the sequence (SEQ ID NO:2).

35. The nucleic acid of claim 28, encoding a fusion protein having the sequence (SEQ ID NO: 3).

36. The nucleic acid of claim 35, having the sequence (SEQ ID NO:4).

37. An expression vector comprising the nucleic acid of claim 28 operatively linked to a control sequence.

38. The expression vector of claim 37, wherein the control sequence is a promoter.

39. The expression vector of claim 38, wherein the promoter is a CMV promoter.

40. The expression vector of claim 37, wherein the vector is a plasmid.

41. The expression vector of claim 40, wherein the vector is an AAV expression plasmid.

42. The expression vector of claim 37, wherein the vector is a viral vector.

43. The expression vector of claim 42, wherein the viral vector is selected from the group consisting of an AAV vector, a Lentiviral vector, a retroviral vector, an Adenoviral vector, and a synthetic viral vector.

44. A cell comprising the nucleic acid of any one of claims 1-27 or the expression vector of any one of claims 37-43.

45. A pharmaceutical composition comprising: (i) a component selected from the group consisting of the fusion protein of any one of claims 1-27, the nucleic acid of any one of claims 28-36, the vector of any one of claims 37-43, or the cell of claim 44; and
(ii) a pharmaceutically acceptable carrier.

46. A method for treating a condition in a mammalian patient, wherein the condition is selected from the group consisting of retinal dystrophy, Stargardt's disease, retinitis pigmentosa, dry age-related macular degeneration (dry AMD), geographic atrophy (advanced stage of dry AMD), wet age-related macular degeneration (wet AMD), glaucoma with or without ocular hypertension, diabetic retinopathy, Bardet-Biedel syndrome, Bassen-Kornzweig syndrome, Best disease, choroidema, gyrate atrophy, congenital amaurosis, refsun syndrome, Usher syndrome, thyroid related eye disease, Grave's disease, a disease associated with retinal pigmented epithelial cells, anterior segment disease, lens disease/cataracts, an eye cup disorder, uveitis, Alzheimer's disease, Huntington's disease, Parkinson's disease, and an olfactory disease;
comprising administering to the patient an effective amount of the fusion protein of any one of claims 1-27, the nucleic acid of any one of claims 28-36, the vector of any one of claims 37-43, the cell of claim 44, or the pharmaceutical composition of claim 45, thereby treating the condition in the patient.

47. The method of claim 46, wherein the condition is an ocular condition and the administration is selected from the group consisting of subretinal injection and intravitreal injection.

48. A method of protecting ocular photoreceptor cells in a patient, comprising administering to the eye of the patient an effective amount of the fusion protein of any one of claims 1-27, the nucleic acid of any one of claims 28-36, the vector of any one of claims 37-43, the cell of claim 44, or the pharmaceutical composition of claim 45, thereby protecting the ocular photoreceptor cells in the patient.

49. The method of claim 48, wherein the administration is selected from the group consisting of subretinal injection and intravitreal injection.

50. The method of any one of claims 46 to 49, wherein the patient is a human patient.

51. A method for producing a fusion protein of any one of claims 1-27, comprising culturing the cell of claim 44 under conditions allowing for expression and secretion of the encoded fusion protein, and isolating the fusion protein from the cell culture.


SEQUENCE LISTING (6 sequences)

SEQ ID NO: 1 - protein, 717 aa, synthetic construct
  SIGNAL 1..24: note = Signal sequence from human albumin
  REGION 1..609: note = MISC_FEATURE - Human albumin (including N-terminal signal peptide)
  REGION 25..609: note = MISC_FEATURE - Human albumin (excluding N-terminal signal peptide)
  REGION 610..717: note = MISC_FEATURE - RdCVFS
MKWVTFISLLFLFSSAYSRGVFRRDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLASLFSGRILIRNNSDQDELDTEAEVSRRLENRLVLLFFGAGACPQCQAFVPILKDFFVRLTDEFYVLRAAQLALVYVSQDSTEEQQDLFLKDMPKKWLFLPFEDDLRR

SEQ ID NO: 2 - DNA, 2154 bp, synthetic construct
  source 1..2154: mol_type = other DNA
atgaagtgggtaacctttatttcccttctttttctctttagctcggcttattccaggggtgtgtttcgtcgagatgcacacaagagtgaggttgctcatcggtttaaagatttgggagaagaaaatttcaaagccttggtgttgattgcctttgctcagtatcttcagcagtgtccatttgaagatcatgtaaaattagtgaatgaagtaactgaatttgcaaaaacatgtgttgctgatgagtcagctgaaaattgtgacaaatcacttcataccctttttggagacaaattatgcacagttgcaactcttcgtgaaacctatggtgaaatggctgactgctgtgcaaaacaagaacctgagagaaatgaatgcttcttgcaacacaaagatgacaacccaaacctcccccgattggtgagaccagaggttgatgtgatgtgcactgcttttcatgacaatgaagagacatttttgaaaaaatacttatatgaaattgccagaagacatccttacttttatgccccggaactccttttctttgctaaaaggtataaagctgcttttacagaatgttgccaagctgctgataaagctgcctgcctgttgccaaagctcgatgaacttcgggatgaagggaaggcttcgtctgccaaacagagactcaagtgtgccagtctccaaaaatttggagaaagagctttcaaagcatgggcagtagctcgcctgagccagagatttcccaaagctgagtttgcagaagtttccaagttagtgacagatcttaccaaagtccacacggaatgctgccatggagatctgcttgaatgtgctgatgacagggcggaccttgccaagtatatctgtgaaaatcaagattcgatctccagtaaactgaaggaatgctgtgaaaaacctctgttggaaaaatcccactgcattgccgaagtggaaaatgatgagatgcctgctgacttgccttcattagctgctgattttgttgaaagtaaggatgtttgcaaaaactatgctgaggcaaaggatgtcttcctgggcatgtttttgtatgaatatgcaagaaggcatcctgattactctgtcgtgctgctgctgagacttgccaagacatatgaaaccactctagagaagtgctgtgccgctgcagatcctcatgaatgctatgccaaagtgttcgatgaatttaaacctcttgtggaagagcctcagaatttaatcaaacaaaattgtgagctttttgagcagcttggagagtacaaattccagaatgcgctattagttcgttacaccaagaaagtaccccaagtgtcaactccaactcttgtagaggtctcaagaaacctaggaaaagtgggcagcaaatgttgtaaacatcctgaagcaaaaagaatgccctgtgcagaagactatctatccgtggtcctgaaccagttatgtgtgttgcatgagaaaacgccagtaagtgacagagtcaccaaatgctgcacagaatccttggtgaacaggcgaccatgcttttcagctctggaagtcgatgaaacatacgttcccaaagagtttaatgctgaaacattcaccttccatgcagatatatgcacactttctgagaaggagagacaaatcaagaaacaaactgcacttgttgagctcgtgaaacacaagcccaaggcaacaaaagagcaactgaaagctgttatggatgatttcgcagcttttgtagagaagtgctgcaaggctgacgataaggagacctgctttgccgaggagggtaaaaaacttgttgctgcaagtcaagctgccttaggcttagccagcctgttcagcggccggatcctgatcaggaacaacagcgaccaggacgagctggacaccgaggccgaagtgagcaggaggctggagaacagactggtgctgctgttctttggcgccggagcctgccctcagtgccaggccttcgtgcccatcctgaaggatttctttgtgcggctgaccgacgagttctacgtgctgagagccgcccagctggccctggtgtatgtgagccaggacagcaccgaggagcagcaggacctgttcctgaaggacatgcccaagaagtggctgttcctgcccttcgaggacgacctgcggagatga

SEQ ID NO: 3 - protein, 732 aa, synthetic construct
  REGION 1..732: note = N-terminal signal sequence from mouse IgK, followed by spacer, followed by RdCVFS, followed by spacer, followed by human albumin at C-terminus.
  SIGNAL 1..21: note = Signal sequence from mouse Igk.
  REGION 22..35: note = MISC_FEATURE - Spacer
  REGION 36..143: note = MISC_FEATURE - RdCVFS
  REGION 144..147: note = MISC_FEATURE - Spacer
  REGION 148..732: note = MISC_FEATURE - Human albumin
METDTLLLWVLLLWVPGSTGDAAQPARRAVRSLVPASLFSGRILIRNNSDQDELDTEAEVSRRLENRLVLLFFGAGACPQCQAFVPILKDFFVRLTDEFYVLRAAQLALVYVSQDSTEEQQDLFLKDMPKKWLFLPFEDDLRRGRDADAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 4 - DNA, 2199 bp, synthetic construct
  misc_feature 1..2199: note = N-terminal signal sequence from mouse IgK, followed by spacer, followed by RdCVFS, followed by spacer, followed by human albumin at C-terminus.
  source 1..2199: mol_type = other DNA
atggagacagacacactcctgctatgggtactgctgctctgggttccaggttccactggtgacgcggcccagccggccaggcgcgccgtacgaagcttggtacccgccagcctgttcagcggccggatcctgatcaggaacaacagcgaccaggacgagctggacaccgaggccgaagtgagcaggaggctggagaacagactggtgctgctgttctttggcgccggagcctgccctcagtgccaggccttcgtgcccatcctgaaggatttctttgtgcggctgaccgacgagttctacgtgctgagagccgcccagctggccctggtgtatgtgagccaggacagcaccgaggagcagcaggacctgttcctgaaggacatgcccaagaagtggctgttcctgcccttcgaggacgacctgcggagaggacgagatgcagatgcacacaagagtgaggttgctcatcggtttaaagatttgggagaagaaaatttcaaagccttggtgttgattgcctttgctcagtatcttcagcagtgtccatttgaagatcatgtaaaattagtgaatgaagtaactgaatttgcaaaaacatgtgttgctgatgagtcagctgaaaattgtgacaaatcacttcataccctttttggagacaaattatgcacagttgcaactcttcgtgaaacctatggtgaaatggctgactgctgtgcaaaacaagaacctgagagaaatgaatgcttcttgcaacacaaagatgacaacccaaacctcccccgattggtgagaccagaggttgatgtgatgtgcactgcttttcatgacaatgaagagacatttttgaaaaaatacttatatgaaattgccagaagacatccttacttttatgccccggaactccttttctttgctaaaaggtataaagctgcttttacagaatgttgccaagctgctgataaagctgcctgcctgttgccaaagctcgatgaacttcgggatgaagggaaggcttcgtctgccaaacagagactcaagtgtgccagtctccaaaaatttggagaaagagctttcaaagcatgggcagtagctcgcctgagccagagatttcccaaagctgagtttgcagaagtttccaagttagtgacagatcttaccaaagtccacacggaatgctgccatggagatctgcttgaatgtgctgatgacagggcggaccttgccaagtatatctgtgaaaatcaagattcgatctccagtaaactgaaggaatgctgtgaaaaacctctgttggaaaaatcccactgcattgccgaagtggaaaatgatgagatgcctgctgacttgccttcattagctgctgattttgttgaaagtaaggatgtttgcaaaaactatgctgaggcaaaggatgtcttcctgggcatgtttttgtatgaatatgcaagaaggcatcctgattactctgtcgtgctgctgctgagacttgccaagacatatgaaaccactctagagaagtgctgtgccgctgcagatcctcatgaatgctatgccaaagtgttcgatgaatttaaacctcttgtggaagagcctcagaatttaatcaaacaaaattgtgagctttttgagcagcttggagagtacaaattccagaatgcgctattagttcgttacaccaagaaagtaccccaagtgtcaactccaactcttgtagaggtctcaagaaacctaggaaaagtgggcagcaaatgttgtaaacatcctgaagcaaaaagaatgccctgtgcagaagactatctatccgtggtcctgaaccagttatgtgtgttgcatgagaaaacgccagtaagtgacagagtcaccaaatgctgcacagaatccttggtgaacaggcgaccatgcttttcagctctggaagtcgatgaaacatacgttcccaaagagtttaatgctgaaacattcaccttccatgcagatatatgcacactttctgagaaggagagacaaatcaagaaacaaactgcacttgttgagctcgtgaaacacaagcccaaggcaacaaaagagcaactgaaagctgttatggatgatttcgcagcttttgtagagaagtgctgcaaggctgacgataaggagacctgctttgccgaggagggtaaaaaacttgttgctgcaagtcaagctgccttaggcttataa

SEQ ID NO: 5 - protein, 109 aa, Homo sapiens
MASLFSGRILIRNNSDQDELDTEAEVSRRLENRLVLLFFGAGACPQCQAFVPILKDFFVRLTDEFYVLRAAQLALVYVSQDSTEEQQDLFLKDMPKKWLFLPFEDDLRR

SEQ ID NO: 6 - protein, 212 aa, Homo sapiens
MASLFSGRILIRNNSDQDELDTEAEVSRRLENRLVLLFFGAGACPQCQAFVPILKDFFVRLTDEFYVLRAAQLALVYVSQDSTEEQQDLFLKDMPKKWLFLPFEDDLRRDLGRQFSVERLPAVVVLKPDGDVLTRDGADEIQRLGTACFANWQEAAEVLDRNFQLPEDLEDQEPRSLTECLRRHKYRVEKAARGGRDPGGGGGEEGGAGGLF